(19) United States
(12) Patent Application Publication
(10) Pub. No.: US 2014/0012119 A1
(43) Pub. Date: Jan. 9, 2014

(54) SKIN ADHESIVE TEMPLATE

(71) Applicants: Chris Geaghan, North Easton, MA (US); James A. Grasfield, Sharon, MA (US)

(72) Inventors: Chris Geaghan, North Easton, MA (US); James A. Grasfield, Sharon, MA (US)

(21) Appl. No.: 13/911,641

(22) Filed: Jun. 6, 2013

Related U.S. Application Data
(60) Provisional application No. 61/656,723, filed on Jun. 7, 2012.

(51) Int. Cl. A61M 5/142; A61B 5/145

(52) U.S. Cl. CPC A61M 5/14248; A61B 5/14532
USPC 600/365; 604/180; 604/179

(57) ABSTRACT

A template sheet is provided for use in the application of a medical grade adhesive to the skin of a user. In one preferred embodiment, the template is fabricated of a flexible plastic material having one or more openings in the sheet and marker openings are provided about the periphery of the one or more openings and through which the skin can be marked with a marking implement. By use of such markers on the skin, the template can be removed after placing the markings on the skin so that adhesive can be applied to the skin within the marked area without need for the template to remain in place during adhesive application. Alternatively, the template may be employed as a mask and the skin adhesive may be applied to the skin while the template is appropriately positioned on the skin of the user. The template may optionally include a strap or belt by which the template can be held in place on a user's body while adhesive is being applied and without requiring the use of the user's hand to hold the template in place. As a further alternative, the template sheet can have adhesive on one or more defined areas of the sheet. After adherence of the adhesive to the skin of a user, the template sheet can be removed to leave the adhesive in place.

### CROSS REFERENCE TO RELATED APPLICATIONS

This application claims priority to U.S. Provisional Appl. No. 61/656,723, filed Jun. 7, 2012, entitled SKIN ADHESIVE TEMPLATE.

### STATEMENT REGARDING FEDERALLY SPONSORED RESEARCH OR DEVELOPMENT

N/A

### BACKGROUND OF THE INVENTION

The present invention relates to continuous glucose monitoring and insulin pumps used by diabetics and more specifically, to a template used to define an area on the skin for the application of a skin adhesive for affixing a continuous glucose monitor transmitter, insulin pump infusion set or sensor to the skin.

Continuous glucose monitors, insulin pump infusion sets or sensors used with such devices are widely employed by diabetics for insulin administration as well as the monitoring of glucose levels. Such devices vary in size and shape. Such devices may be affixed to the skin of a user via the use of a skin adhesive. When affixing such devices to the skin of a user, it is preferable to apply a medical grade adhesive of an appropriate size and shape so that adhesive on the skin does not extend beyond the area occupied by the applied device.

It would therefore be helpful to provide a convenient and easy to use aid which allows a user to apply the adhesive to the skin in a position, size and shape prior to the affixation of a glucose monitor, infusion set or sensor.

### BRIEF SUMMARY OF THE INVENTION

The present invention comprises a template or stencil used in the application of a medical grade adhesive to the skin. In one embodiment, the template is fabricated of a flexible plastic material which may be die cut or molded in a desired size and shape. Other durable and flexible sheet material can be used in alternative embodiments. The template according to the invention has a central opening and marker openings are provided in the template sheet about the periphery of the central opening and through which the skin can be marked with a non-toxic marker pen or other marking implement. By use of the markings on the skin, the template can be removed after placing the marks on the skin so that adhesive can be applied to the skin within the marked area without need for the template to remain in place during adhesive application. The size and shape of the central opening can vary to suit the specifications of monitoring and infusion devices employed by the user. It is contemplated that one or more openings can be provided in the sheet to define multiple areas where adhesive is to be applied, rather than a single central opening.

Alternatively, the template may be employed as a mask and the skin adhesive may be applied to the skin while the template is appropriately positioned on the skin of the user. The template may optionally include a strap or belt by which the template can be held in place on a user's body while adhesive is being applied and without requiring the use of the user's hand to hold the template in place. The template may optionally be coated with a minimally adhering medical grade adhesive on one face by which the template may be held in place on a user's body while adhesive is being applied and/or marks are made on the skin without requiring the use of the user's hand or a belt to hold the template in place.

According to another aspect of the invention, a pre-printed non-toxic ink pattern may be transferred to the user's skin from the template by applying the template to the skin with light pressure and then removing said template leaving the desired ink pattern on the user's skin. Adhesive can subsequently be applied to the marked area or areas on the skin by the user, and a continuous glucose monitor transmitter, insulin pump infusion set or other medical device may then be affixed to the user's skin by said adhesive area or areas.

According to another aspect of the invention, a medical grade adhesive can be applied to one or more areas of the template sheet which is of a construction and material to be peelable from the adhesive. The template in this embodiment is applied to the skin of a user at a position where adhesive is desired and the adhesive adheres to the user's skin at that position. The template sheet is thereafter peeled away or otherwise removed from the user's skin to leave the adhesive in place for receipt of a continuous glucose monitor transmitter, insulin pump infusion set or other medical device.

### BRIEF DESCRIPTION OF THE SEVERAL VIEWS OF THE DRAWINGS

Other features, aspects and advantages of the presently disclosed template will be apparent to those of ordinary skill in the art from the drawing and the detailed description of the invention that follows.

FIG. 1 shows a plan view of one embodiment of a template according to the invention;

FIG. 2 shows a pictorial view of the template of FIG. 1 in use;

FIG. 3 is a pictorial view of the markings provided by use of the template of FIG. 1;

FIG. 4 is a pictorial view showing the template of FIG. 1 retained on a user's body;

FIG. 5 is a pictorial view of another embodiment of a template according to the invention;

FIG. 6 is a pictorial view of the template of FIG. 5 placed on a user's skin;

FIG. 7 is a pictorial view of the embodiment of FIG. 6 in use;

FIG. 8 is a pictorial view of the markings and peelaway of the template of FIG. 5;

FIG. 9 is a pictorial view showing removal of a backing sheet from a membrane of another embodiment;

FIG. 10 is a pictorial view showing the membrane of FIG. 9 affixed to a user's skin;

FIG. 11 is a pictorial view showing removal of the membrane of FIG. 9 from a user's skin;

FIG. 12 is a pictorial view showing the markings provided by the membrane of FIG. 9;

FIG. 13 is a pictorial view of a membrane of another embodiment prior to its application on a user's skin;

FIG. 14 is a pictorial view showing application of the membrane of FIG. 13;

FIG. 15 is a pictorial view showing removal of the membrane of FIG. 13 from a user's skin; and

FIG. 16 is a pictorial view showing the adhesive pattern on the user's skin left by the removed membrane of FIG. 13.

### DETAILED DESCRIPTION OF THE INVENTION

The disclosure of U.S. Provisional Application 61/656,723 is incorporated herein in its entirety.

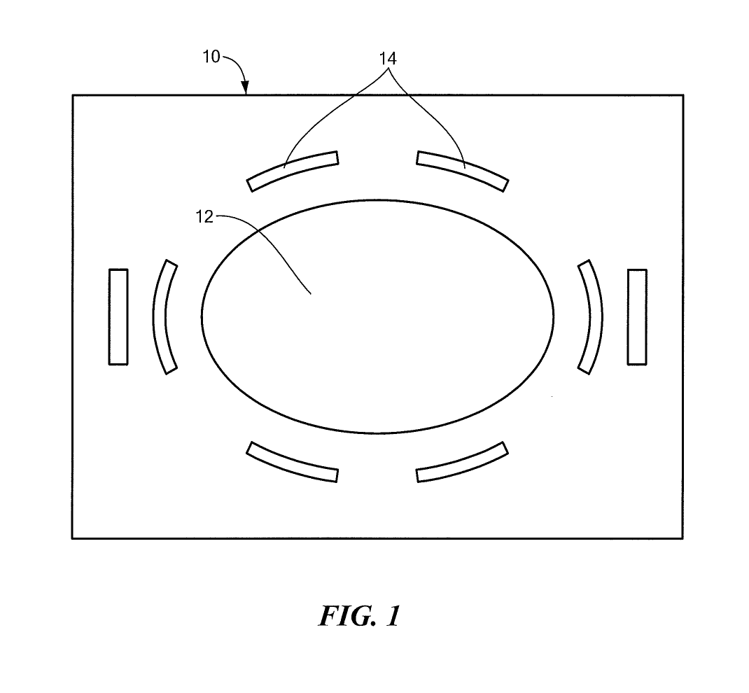

Referring to FIG. 1 a template sheet 10 is shown which is typically made of a plastic material such as a Nylon material which is relatively stiff but which has some flexibility to be flexed onto a user's arm, leg or other body portion. The sheet 10 is thin and typically about ⅛ of an inch thick or less. The sheet 10 includes a central opening 12 which in the illustrated embodiment is of oval shape. In the exemplary embodiment, a plurality of arc shaped openings or cutouts 14 are provided around the central opening 12 which are disposed in spaced relation about the periphery of the central opening. Rectangular openings or cutouts 16 are optionally provided on opposing side ends of the sheet 10.

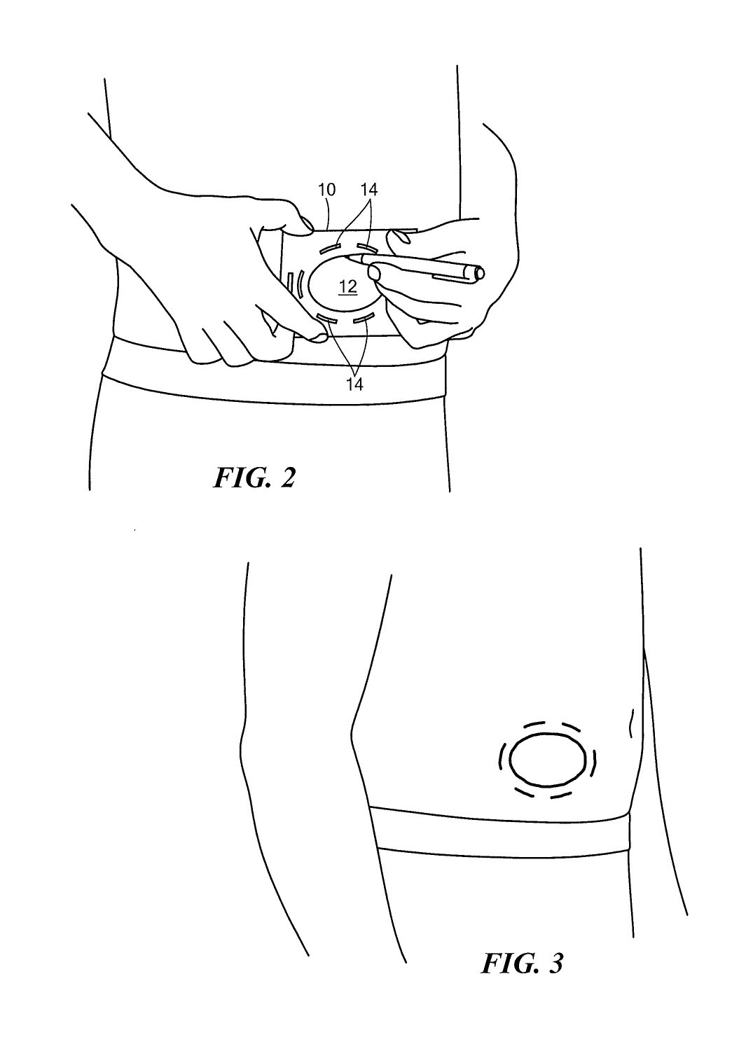

The central opening 12 is used to define an area of the skin of a user to which a medical grade adhesive is to be applied. The arc shaped cutouts 14 are for the purpose of marking the skin with a marker pen or other appropriate implement to clearly identify the area of the skin to which the adhesive is to be applied as shown in FIG. 2. Once marked, the template sheet 10 can be removed as shown in FIG. 3 and the adhesive thereafter applied to the skin in the area within the markings. Skin adhesives such as MASTISOL® Liquid Adhesive produced by Ferndale Laboratories, Ferndale, Mich., SKIN-TAC™ Liquid Adhesive produced by Torbot Group Inc., Cranston, R.I. or any other suitable adhesive may be employed for affixing the glucose monitor, infusion set, sensor or other device to the user's skin.

It will be appreciated that the template sheet 10 can be removed following marking of the skin as described above or alternatively, the template sheet 10 can be held on the skin and used as a mask until after the adhesive is applied through the central opening. Following application of the adhesive to the skin, the template sheet 10 is removed and the continuous glucose monitor transmitter, insulin pump infusion set or sensor is affixed to the skin via the adhesive. When using the template sheet 10 as a mask, it will be recognized that there is no need for marking of the skin since the relevant area to which the adhesive is to be applied is defined by the central opening.

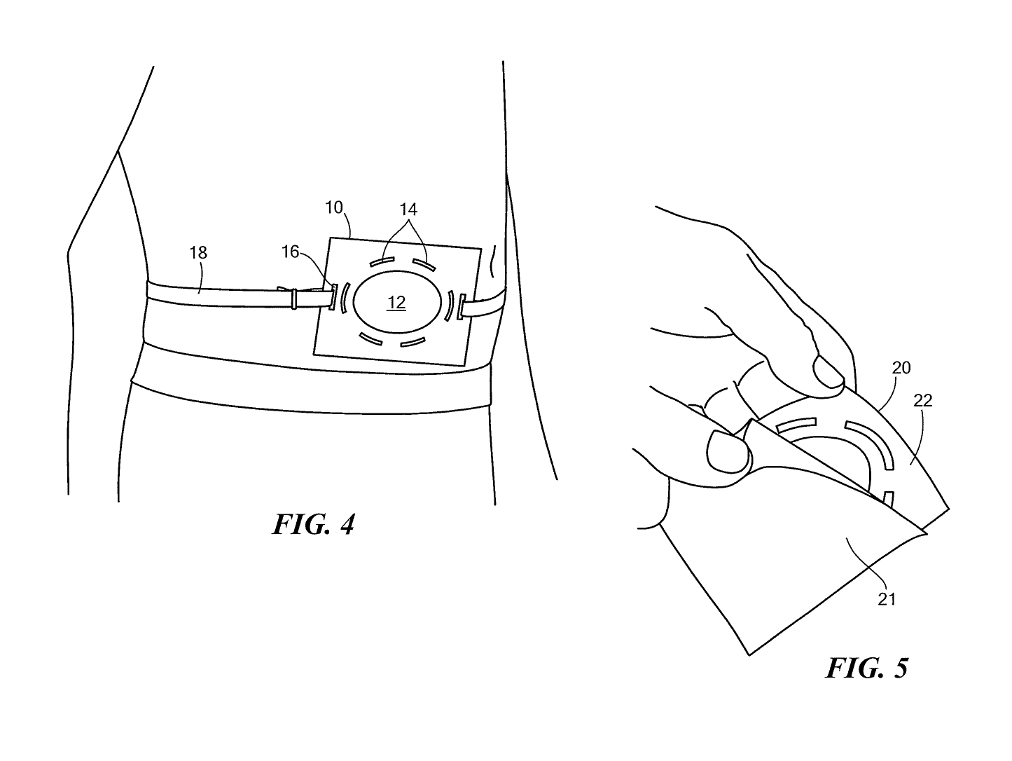

The template sheet 10 can be held in place on the skin by the use of an optional strap or belt 18 that can be secured via the rectangular cutouts 16 at opposing side ends of the template sheet 10 as shown in FIG. 4. The strap 18 can include an adjustable buckle, clasp or hook and loop fasteners to secure the template 10 to the user's body during use in marking and/or applying adhesive to the skin.

The template sheet can be manufactured in different configurations having dimensions and shaped openings to accommodate the specifications of different insulin pump infusion sets, continuous glucose monitor transmitters, sensors, or other medical devices or appliances.

In another embodiment of the present invention a template sheet with the pattern of a particular apparatus as previously described in the first embodiment may be fabricated with an adhesive backing on one face of the template sheet and a backing sheet of similar size covering the adhesive. The template sheet may be made of a thin and flexible film or membrane that is die cut or molded in a desired shape and size. Other durable and flexible sheet material may be used in alternative embodiments. The adhesive applied to the film or membrane is medically safe but has light adhesion that will only stick temporarily. In this embodiment the template sheet may be held in place temporarily on the user's skin while the user marks the stencil pattern with an appropriate marker. The self-adhering template sheet enables the user to apply the template sheet and mark the pattern for the apparatus on the skin with a single hand, making this particularly advantageous when the user applies the template sheet to his/her arm. The self-adhering template sheet also stabilizes the stencil during the marking process to permit the user to easily and more precisely mark the pattern on the user's skin.

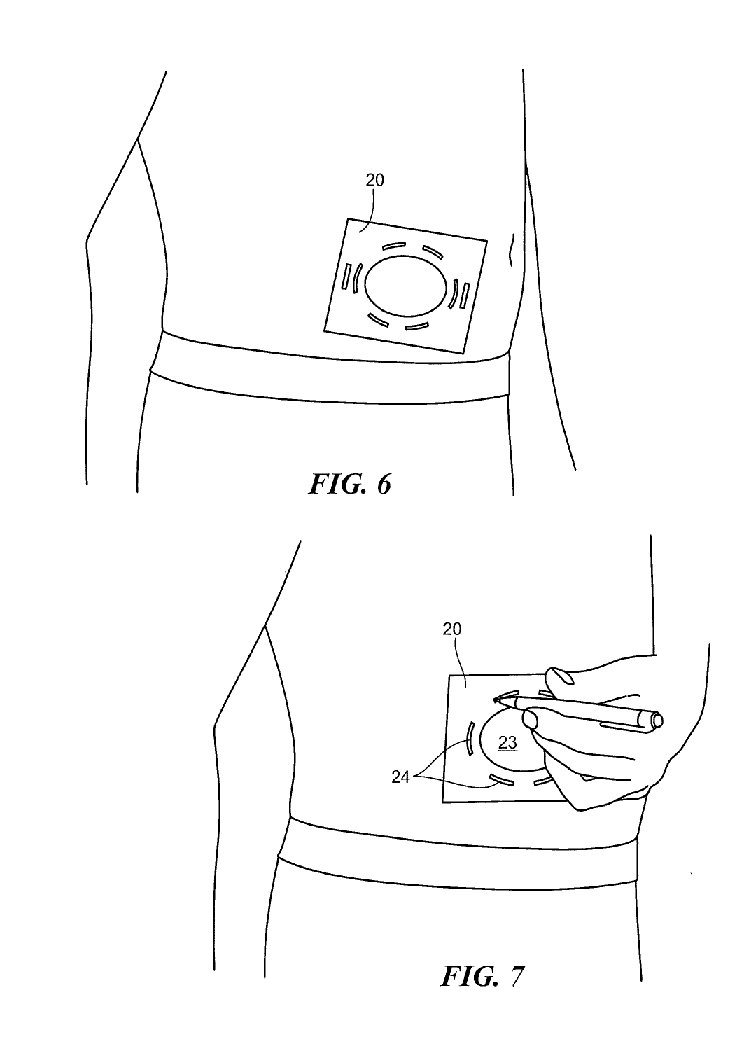

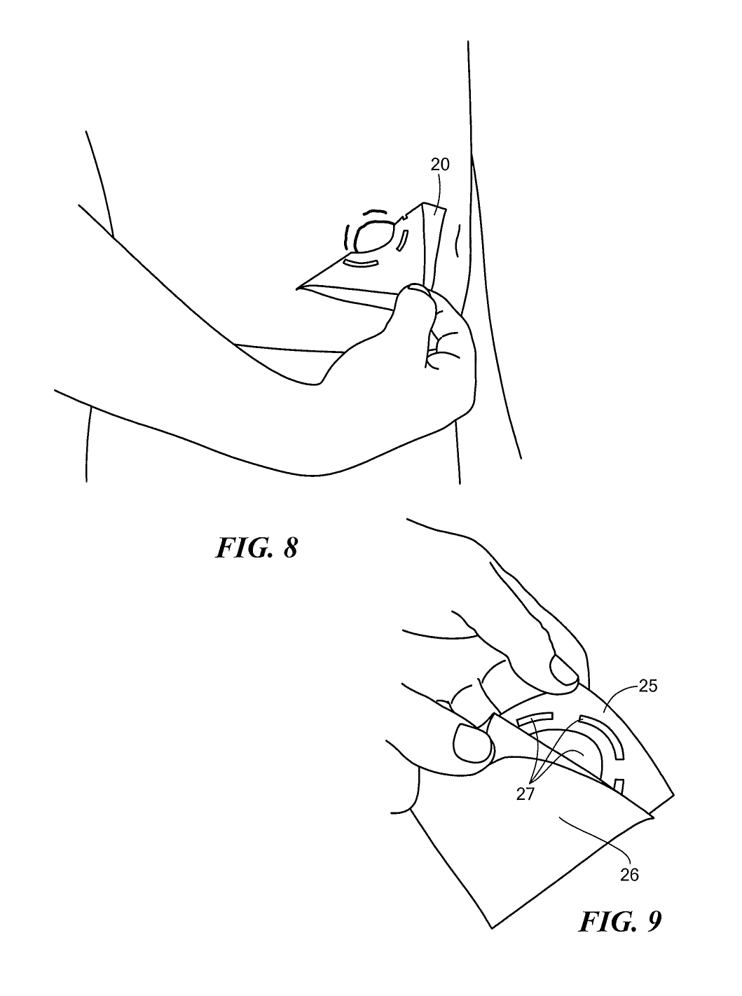

Reference is now made to FIG. 5 which shows the template sheet 20 prior to its application at an exemplary site for usage on the user's abdomen. To apply the template sheet 20 to the user's skin, the user peels the backing sheet 21 from the template sheet 20 exposing the temporary adhesive 22. The user then places the template sheet 20 against his/her skin in the desired location with light pressure to adhere the template sheet 20 to the skin as shown in FIG. 6. As shown in FIG. 7, the user then marks the pattern of the stencil via central opening 23 and arc shaped openings or cutouts 24 for the specific apparatus on the skin using an appropriate marker or pen. Once the marks have been completed, the user peels the template sheet 20 off the skin leaving only the marked pattern on the skin and no temporary adhesive residue as shown in FIG. 8. The user can then apply medical adhesive as previously described to the marked areas on the skin and subsequently apply the apparatus to the skin via the defined area of medical adhesive. The removed template sheet 20 may be disposable.

Alternatively, the self-adhering template sheet 20 of the embodiment described above may be employed as a mask, and the medical adhesive may be applied to the skin using the central opening 23 in the template sheet 20 while it is self-adhered to the skin in the appropriate location. Once the user has applied the medical adhesive to the skin through the mask, the template sheet 20 can be removed from the user's skin, leaving the pattern of the adhesive for the apparatus on the user's skin. The user then applies the apparatus to the adhesive pattern to affix it to the user's skin. When using the template sheet 20 as a mask, it will be recognized that there is no need for marking of the skin since the relevant area to which the adhesive is to be applied is defined by the central opening. The removed mask may be disposable.

In another embodiment of the present invention a printed pattern similar in size, configuration, and shape to the previously described stencil may be transferred to the user's skin via a flexible membrane or film applied to the skin. The membrane may be fabricated of a flexible plastic material which may be die cut or molded in a desired shape and size. Other durable and flexible sheet material may be used in alternative embodiments. An ink-based pattern is applied to the membrane on one face and then covered by a backing sheet which keeps the ink from drying.

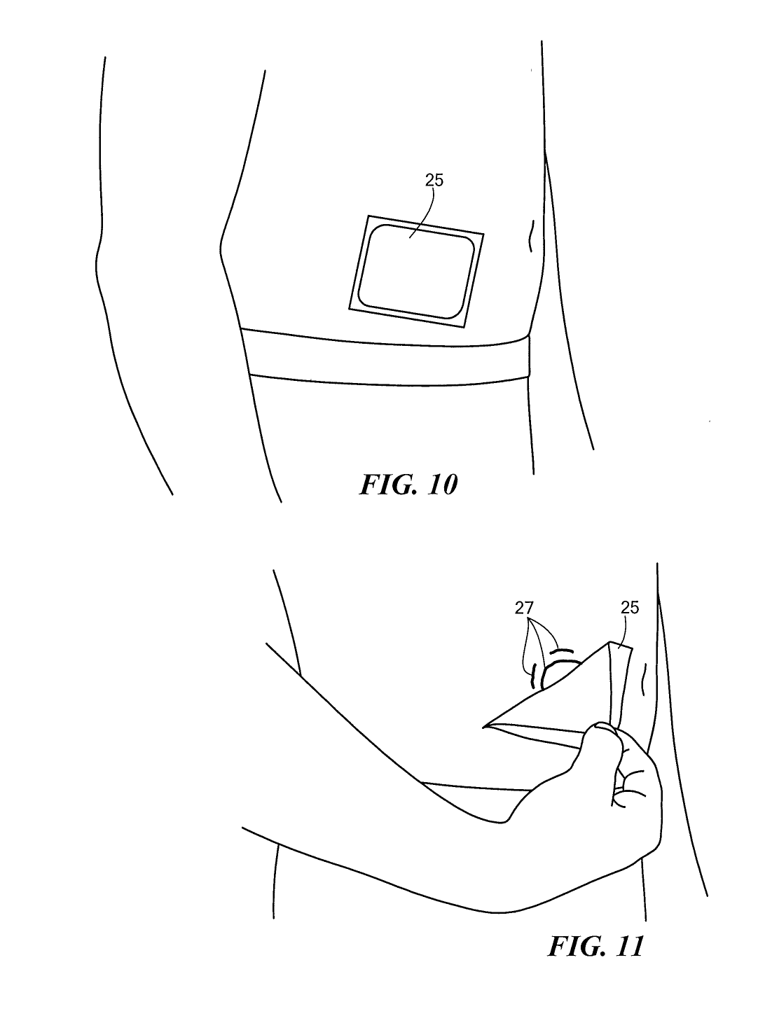

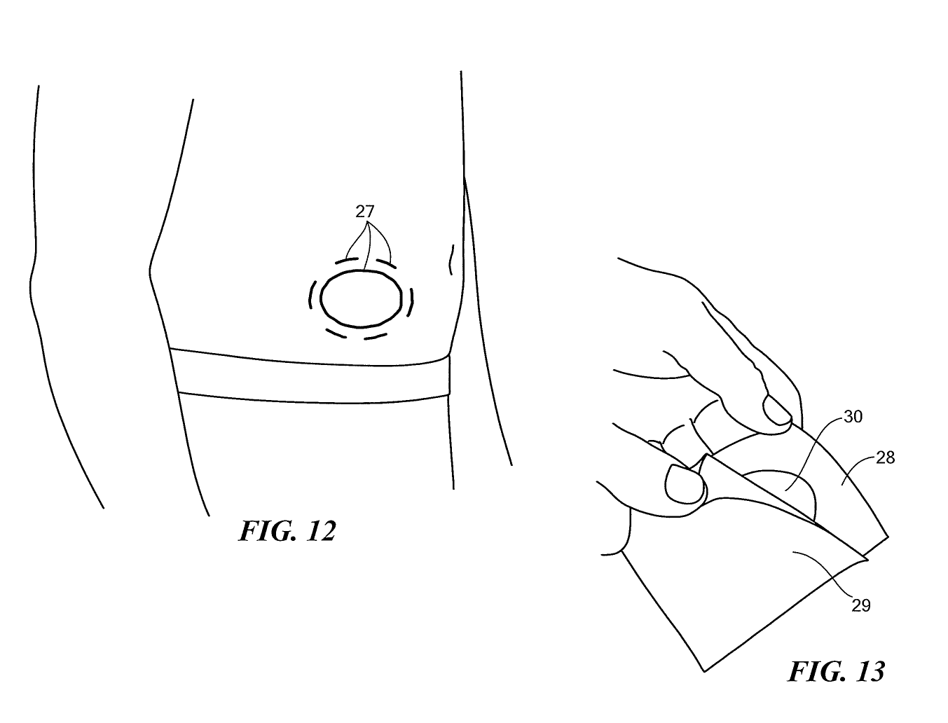

Reference is made to FIG. 9 which shows the membrane 25 prior to its application at an exemplary site for usage on the user's abdomen. To apply the pattern, the user removes the backing sheet 26 from the membrane 25 which exposes the wet non-toxic ink pattern 27 of the appropriate size and shape of a continuous glucose monitor transmitter, insulin pump infusion set or sensor to be affixed to the user's skin. The user places the membrane with the pre-printed ink pattern 27 on the skin in the desired location as shown in FIG. 10, applies light pressure to the entire surface of the membrane 25 against the skin, and then peels the membrane 25 from the skin as shown in FIG. 11. The ink pattern 27 transfers to the user's skin delineating the precise location for the application of the medical adhesive necessary to affix the desired continuous glucose monitor transmitter, insulin pump infusion set or sensor as shown in FIG. 12. The user can then apply a medical adhesive to the area defined by the transferred pattern 27, and then affix the apparatus to the skin via the adhesive. It can be appreciated that in this embodiment the user can apply the pattern with a single hand, making this embodiment particularly advantageous to application on the user's arms.

In another embodiment of the present invention a pre-defined pattern of medical adhesive similar in size, configuration, and shape to the previously described template sheet may be transferred to the user's skin via a flexible membrane or film applied to the skin. The membrane may be fabricated of a flexible plastic material which may be die cut or molded in a desired shape and size. Other durable and flexible sheet material may be used in alternative embodiments. Medical grade adhesive may be applied to one surface of the membrane in a pattern of the appropriate size and shape of a continuous glucose monitor transmitter, insulin pump infusion set or sensor to be affixed to the user's skin. A backing sheet covers the adhesive to keep it from drying and from sticking to any surface or object.

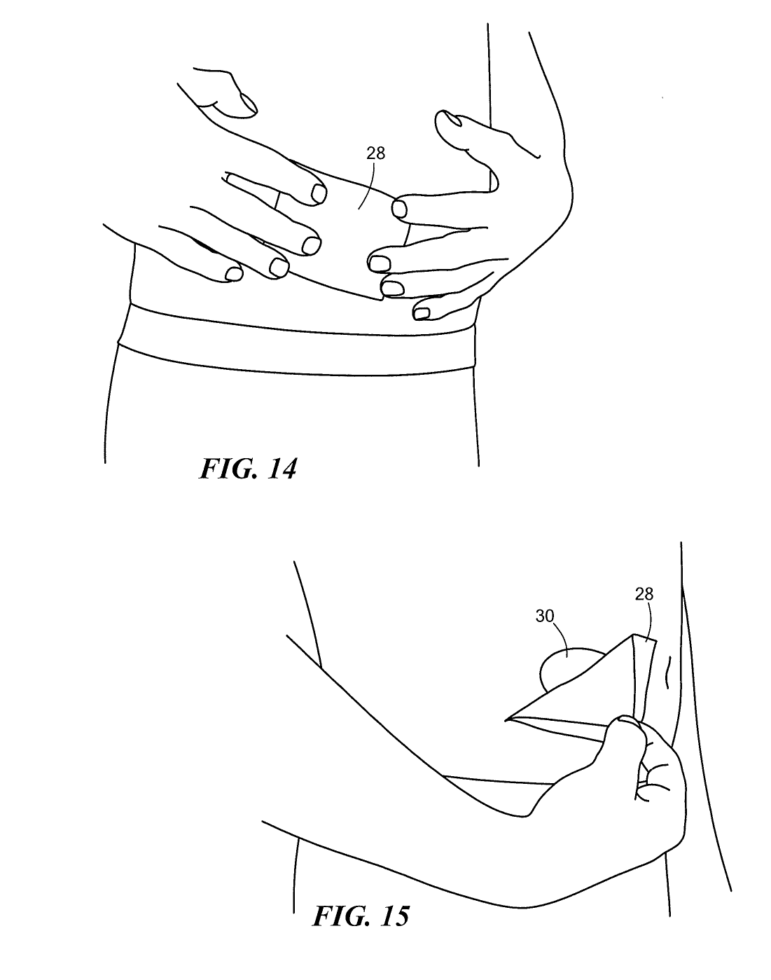

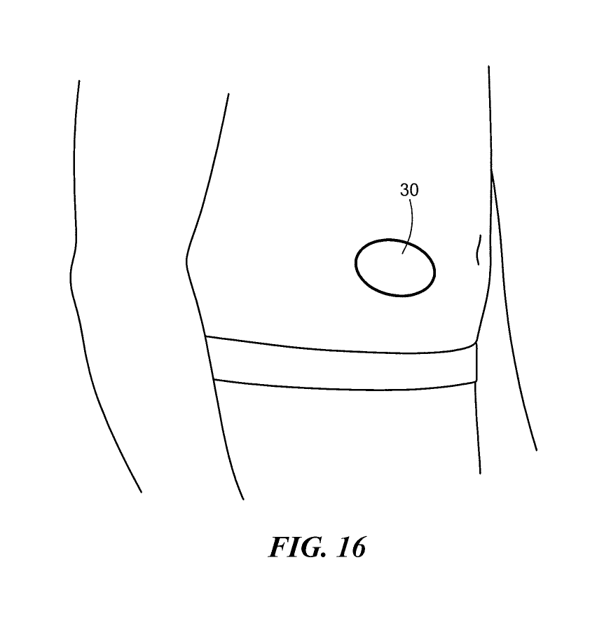

Reference is made to FIG. 13 which shows the membrane 28 prior to its application at an exemplary site for usage on the user's abdomen. To apply the adhesive to the user's skin, the user removes the backing sheet 29 from the membrane 28 which exposes the pattern of medical grade adhesive 30 for the appropriate continuous glucose monitor transmitter, insulin pump infusion set or sensor. The user places the surface of the membrane 28 with the adhesive pattern 30 against his/her skin in the desired location and applies light pressure across the surface of the membrane 28 to adhere the adhesive pattern 30 to the skin as shown in FIG. 14. The user then peels the membrane 28 away from the skin as shown in FIG. 15 leaving the adhesive pattern 30 on the user's skin for the appropriate continuous glucose monitor transmitter, insulin pump infusion set or sensor as shown in FIG. 16. The user then applies the continuous glucose monitor transmitter, insulin pump infusion set or sensor directly to the surface of the adhesive pattern 30 affixed to the skin with light pressure to attach the apparatus to the skin. This embodiment is particularly advantageous because it eliminates the process of having to define and mark the target area first and then in a second step apply the medical adhesive before affixing the apparatus. This embodiment also has the advantage of enabling the user to apply the adhesive and/or apparatus with a single hand. This is particularly beneficial if the user is self-applying the apparatus to his/her arm.

Alternatively, the adhesive pattern 30 may be applied to the apparatus directly first via the membrane 28. After the membrane 28 has been removed by the user, the user may affix the apparatus to the user's skin in the desired location by applying light pressure to the apparatus onto the surface of the user's skin. The pre-applied adhesive pattern 30 affixes the apparatus to the user's skin. In another alternative embodiment, the adhesive pattern 30 may be tinted with a coloring agent to assist the user in seeing the adhesive pattern 30 on the skin prior to applying the apparatus to the skin.

It should be understood that modification to and variations of the above-described invention may be made without departing from the inventive concepts disclosed herein. For example, the invention is not limited to use with continuous glucose monitor transmitters and insulin pump infusion sets but is equally applicable to other medical devices and appliances including electrodes, sensors, external pacemakers, defibrillators and the like. Accordingly, the invention should not be viewed as limited except by the scope and spirit of the appended claims.

What is claimed is:

1. A template for application of a medical grade adhesive to the skin of a user, the template comprising:
a sheet of flexible material that is sufficiently flexible to be placed in conforming relation on the skin of a user;
the sheet having at least one area of predetermined size and shape for containing the medical grade adhesive which is adherent to the skin of a user;
the sheet being removable from the skin of a user to leave the adhesive on the skin of the user for subsequent attachment of a medical device.

2. A template for application of a medical grade adhesive to the skin of a user, the template comprising:
a sheet of flexible material that is sufficiently flexible to be placed in conforming relation on the skin of a user; the sheet having at least one area for defining an adhesive application position;
the medical grade adhesive being applyable to the skin of a user at the position defined by at least one area of the sheet as placed on the skin of the user;
the sheet being removeable from the skin of a user to leave the adhesive on the user's skin for subsequent attachment of a medical device.

3. The template of claim 1 wherein the at least one area of the sheet has at least one opening to receive the medical grade adhesive thereon.

4. The template of claim 1 wherein the at least one area of the sheet has the medical grade adhesive thereon.

5. The template of claim 1 wherein the at least one area of the sheet has a central opening;
and including a plurality of peripheral openings disposed in spaced relation about the periphery of the central opening;
the central opening being sized to receive the medical grade adhesive for subsequent attachment of a medical device;
the plurality of peripheral openings being sized to receive a marking implement for applying markings to the skin of a user at the position of the plurality of openings.

6. The template of claim 1 wherein the template sheet is a flexible plastic sheet.

7. The template of claim 1 wherein the sheet is generally rectangular.

8. The template of claim 4 wherein the central opening has a generally oval shape.

9. The template of claim 1 wherein the peripheral openings have a generally arcuate shape.

10. The template of claim 4 wherein the sheet has opposed side ends and first and second cutouts within the sheet sized to receive a strap are respective side ends.

11. The template of claim 9 further including a strap portion extending through the first cutout and secured to the sheet at the respective side end of the sheet.

12. The template of claim 9 further including first and second strap portions extending through the first and second cutouts at respective side ends of the sheet.

13. The template of claim 8 further including fasteners at respective ends of the first and second strap portions for securing the first and second strap portions to each other.

14. The template of claim 12 wherein one of the strap portions is adjustable in length.

15. The template of claim 12 wherein the fasteners are hook and loop fasteners.

16. The template of claim 12 wherein the fasteners are buckle fasteners.

17. A method to facilitate mounting of a medical device to the skin of a user, the method comprising:
providing a template sheet having at least one area of predetermined size and shape and containing a medical grade adhesive in the at least one area;
applying the template sheet to the skin of a user to adhere the adhesive to the skin of the user; and
removing the template sheet from the skin of the user to leave the adhesive in place on the user's skin for subsequent attachment of a medical device.

18. A method to facilitate mounting of a medical device to the skin of a user, the method comprising:
applying a template sheet to the skin of a user, the sheet having a central area of predetermined size and shape and a plurality of cutout slots disposed around the central opening and sized to receive a marking instrument;
marking the skin through the plurality of cutout slots using the marking implement;
removing the sheet from the skin; and
applying a medical grade adhesive to the central area of the template sheet.

19. The method of claim 17 further including the step of disposing the device in contact with the medical grade adhesive to adhere the device to the skin of the user.

20. The method of claim 17 further including between the applying and marking steps the step of securing the sheet to a body part of the user via first and second strap portions extending through first and second cutouts disposed at opposing side ends of the sheet.

21. A method to facilitate mounting of a device to the skin of a user, the device being one of a glucose monitor, an infusion set or a sensor, the method comprising:
applying a template sheet to the skin of the user, the template sheet having a central opening;
securing the template sheet to a body part of the user via at least one strap secured to the template sheet at at least one side end thereof;
applying a medical grade adhesive to the skin within an area defined by the central opening in the sheet; and
removing the sheet from the body part of the user.

22. The method of claim 20 further including the step of disposing the device in contact with the medical grade adhesive to adhere the device to the skin of the user.

23. The method of claim 20 wherein the step of securing the sheet to the body part of the user comprises the step of securing the sheet to the body part of the user via fasteners on first and second strap portions, the strap portions secured to the sheet through first and second cutouts at opposing side ends of the sheet.
